(19) United States
(12) Patent Application Publication
(10) Pub. No.: US 2009/0108092 A1
(43) Pub. Date: Apr. 30, 2009

(54) Accessory

(76) Inventor: Te-En Liu, Taichung City (TW)

Correspondence Address: WHYTE HIRSCHBOECK DUDEK S C;INTELLECTUAL PROPERTY DEPARTMENT, 555 EAST WELLS STREET, SUITE 1900, MILWAUKEE, WI, 53202, US

(21) Appl. No.: 11/877,763

(22) Filed: Oct. 24, 2007

(51) Int. Cl. A61L 9/12

(52) U.S. Cl. 239/45; 239/44

(57) ABSTRACT

An accessory element includes: an enclosure made of an airproof material, defining a chamber therein, having two opposite sides, and formed with a loading hole for loading of a perfume into the chamber and for releasing the fragrance of the perfume therefrom; and an absorbent member enclosed by the enclosure for absorbing the perfume loaded into the chamber. The absorbent member cooperates with the enclosure to form into an integral piece that is formed with a string-hole extending through the opposite sides of the enclosure and the absorbent member.

### BACKGROUND OF THE INVENTION

1. Field of the Invention

This invention relates to an accessory element, more particularly to an accessory element including an enclosure enclosing an absorbent member and formed with a loading hole.

2. Description of the Related Art

A conventional accessory usually includes a supporting frame and an absorbent member framed by the supporting frame for absorbing a perfume. Since the absorbent member is almost entirely exposed to the ambient air, the perfume absorbed in the absorbent member is likely to be consumed very quickly.

### SUMMARY OF THE INVENTION

Therefore, the object of the present invention is to provide an accessory that can overcome the aforesaid drawback associated with the prior art.

According to the present invention, there is provided an accessory element capable of releasing the fragrance of a perfume stored therein. The accessory element comprises: an enclosure made of an airproof material, defining a chamber therein, having two opposite sides, and formed with a loading hole for loading of the perfume into the chamber and for releasing the fragrance of the perfume therefrom; and an absorbent member enclosed by the enclosure for absorbing the perfume loaded into the chamber. The absorbent member cooperates with the enclosure to form into an integral piece that is formed with a string-hole extending through the opposite sides of the enclosure and the absorbent member.

### BRIEF DESCRIPTION OF THE DRAWINGS

In drawings which illustrate an embodiment of the invention,

FIG. 1 is a perspective view of the preferred embodiment of an accessory element according to this invention;

FIG. 2 is a sectional view of the preferred embodiment;

FIG. 3 is a perspective view illustrating how a perfume is loaded into a chamber of an enclosure of the preferred embodiment;

FIG. 4 is a perspective partly cutaway view of an accessory made from a plurality of the accessory elements of the preferred embodiment according to this invention; and

FIG. 5 is a fragmentary perspective view illustrating how the accessory elements of the preferred embodiment can be assembled to form the accessory using a pair of strings.

### DETAILED DESCRIPTION OF THE PREFERRED EMBODIMENT

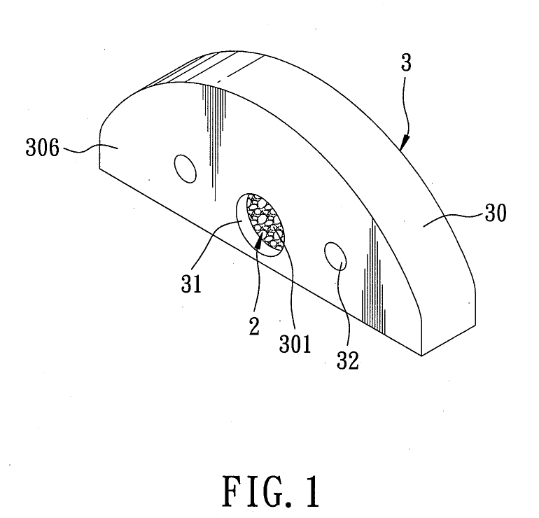

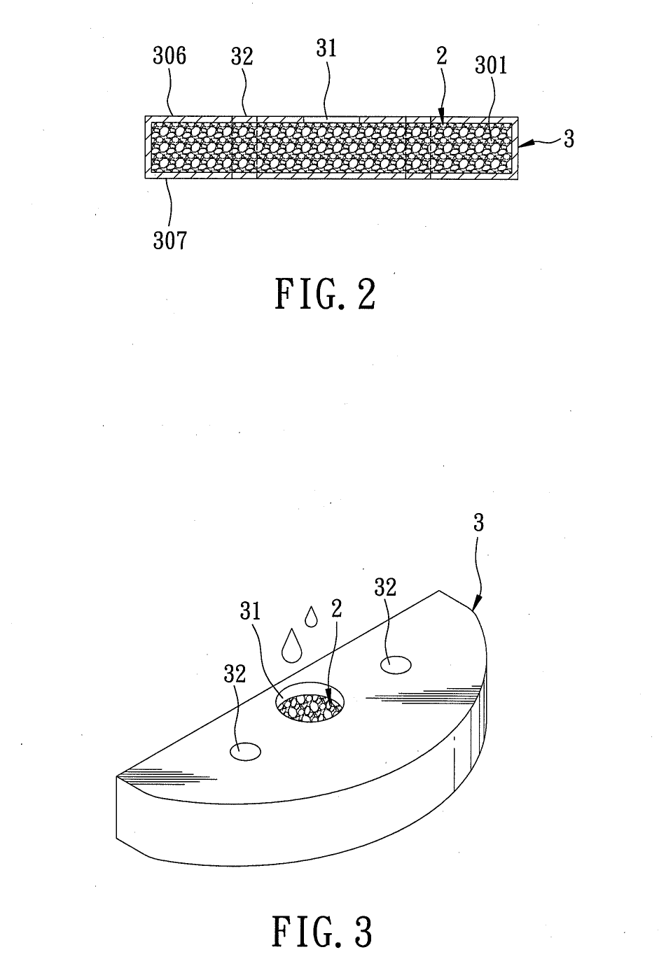

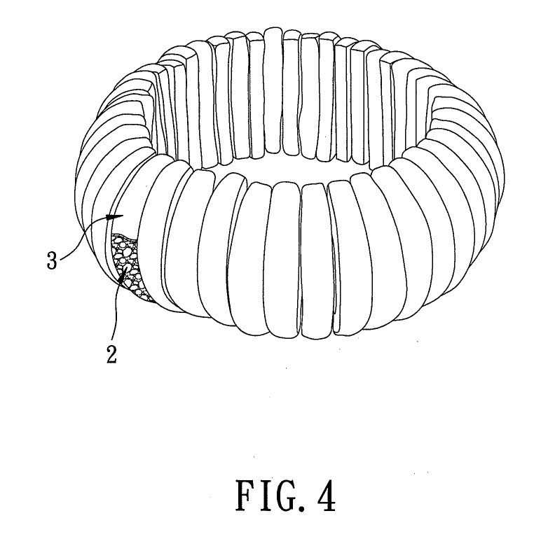

FIGS. 1 to 3 illustrate the preferred embodiment of an accessory element 3 according to this invention.

The accessory element 3 is capable of releasing the fragrance of a perfume stored therein, and includes: an enclosure 30 made of an airproof material, defining a chamber 301 therein, having two opposite sides 306, 307, and formed with a loading hole 31 for loading of the perfume into the chamber 301 and for releasing the fragrance of the perfume therefrom; and an absorbent member 2 enclosed by the enclosure 30 for absorbing the perfume loaded into the chamber 301. The absorbent member 2 cooperates with the enclosure 30 to form into an integral piece that is formed with two string-holes 32 extending through the two opposite sides 306, 307 of the enclosure 30 and the absorbent member 2, thereby permitting extension of strings therethrough.

In this embodiment, the absorbent member 2 is made from a mixture composed of clay and alumina powder, and is made by disposing the mixture in a plaster mold, followed by drying and sintering the mixture so as to form a porous structure.

Preferably, the enclosure 30 is made from colored glaze.

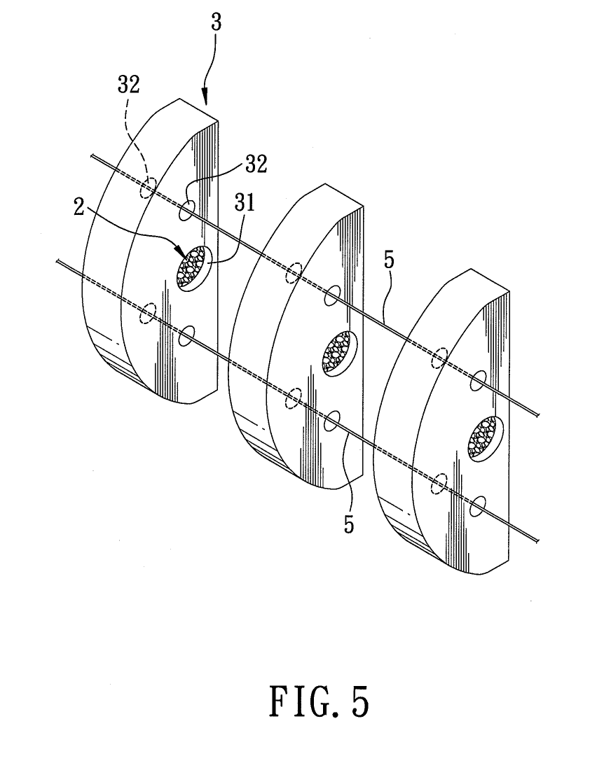

FIGS. 4 and 5 illustrate an accessory which is made from a plurality of the accessory elements 3. A pair of strings 5 extend through the string-holes 32 in the accessory elements 3 (FIG. 5) to form various articles, such as an ankle chain, an earring and a ring, etc.

In use, the perfume is supplied through the loading hole 31 so as to impregnate the absorbent member 2. As such, the fragrance of the perfume can be released to the ambient air from the loading hole 31. Since the enclosure 30 encloses almost an entire surface of the absorbent member 2, the rate of consumption of the perfume can be considerably reduced.

With the inclusion of the enclosure 30 in the accessory element 3 of this invention, the aforesaid drawback associated with the conventional accessory can be eliminated.

With the invention thus explained, it is apparent that various modifications and variations can be made without departing from the spirit of the present invention. It is therefore intended that the invention be limited only as recited in the appended claims.

What is claimed is:

1. An accessory element capable of releasing the fragrance of a perfume stored therein, comprising:
an enclosure made of an airproof material, defining a chamber therein, having two opposite sides, and formed with a loading hole for loading of the perfume into said chamber and for releasing the fragrance of the perfume therefrom; and
an absorbent member enclosed by said enclosure for absorbing the perfume loaded into said chamber;
wherein said absorbent member cooperates with said enclosure to form into an integral piece that is formed with a string-hole extending through said opposite sides of said enclosure and said absorbent member.

2. The accessory element of claim 1, wherein said absorbent member is made from a mixture composed of clay and alumina powder.

3. The accessory element of claim 1, wherein said enclosure is made from colored glaze.

4. An accessory capable of releasing the fragrance of a perfume stored therein, comprising:
a plurality of accessory elements, each including
an enclosure made of an airproof material, defining a chamber therein, having two opposite sides, and formed with a loading hole for loading of the perfume into said chamber, and
an absorbent member enclosed by said enclosure for absorbing the perfume loaded into said chamber, said absorbent member cooperating with said enclosure to form into an integral piece that is formed with a string-hole extending through said opposite sides of said enclosure and said absorbent member; and
a string extending through said string-holes in said accessory elements.
